(19) United States
(12) Patent Application Publication
(10) Pub. No.: US 2014/0113354 A1
(43) Pub. Date: Apr. 24, 2014

(54) COMPOSITION CONTAINING MICROBIAL ZEAXANTHIN AND PREPARATION THEREOF

(71) Applicant: VIGENENT INC., Hsinchu City (TW)

(72) Inventor: Ken-Shwo DAI, Hsinchu City (TW)

(73) Assignee: VIGENENT INC., Hsinchu City (TW)

(21) Appl. No.: 14/139,852

(22) Filed: Dec. 23, 2013

Related U.S. Application Data
(62) Division of application No. 12/904,079, filed on Oct. 13, 2010.

(51) Int. Cl. C12N 1/20; C12R 1/01

(52) U.S. Cl. CPC C12N 1/20; C12R 1/01
USPC 435/252.1

(57) ABSTRACT

The present invention relates to a composition for prevention and/or treatment of human skin alteration or disease comprising an extract of marine bacteria selected from Flavobacteriaceae, wherein the extract contains an effective amount of zeaxanthin. The present invention also relates to a method for preparing the above composition, comprising: (a) culturing marine bacteria selected from Flavobacteriaceae, more specifically, from Olleya marilimosa, in a liquid culturing medium to form pigments containing zeaxanthin; (b) separating cell mass of the marine bacteria with the pigments and the liquid culturing medium and collect the cell mass to obtain the above composition, and (c) optionally mixing the cell mass and a carrier.

### CROSS-REFERENCE TO RELATED APPLICATION

This is a divisional application of U.S. Patent application Ser. No. 12/904079 filed Oct. 13, 2010 entitled “Composition containing microbial zeaxanthin and preparation thereof” which is incorporated herein by reference.

### BACKGROUND OF THE INVENTION

1. Field of the Invention

This invention provides a composition for prevention and/or treatment of human skin alteration or disease comprising an extract of marine bacteria selected from Flavobacteriaceae, more specifically, from Olleya marilimosa, wherein the extract contains an effective amount of zeaxanthin. The composition can be used in the fields of nutraceutical, food additives, cosmetics/skin care and pharmaceuticals.

2. Description of the Related Art

Carotenoids are a group of lipophilic pigments found abundant in plants, algae, and bacteria. These natural pigments are responsible for the different colors (e.g., red, yellow and orange) shown in the living organisms. Over 600 carotenoids have been identified and are structurally divided into carotenes (hydrocarbon) and xanthophylls (oxygenated carotenes). Xanthophylls are yellow color pigments. With the addition of oxygen atoms, xanthophylls (e.g., lutein, neoxanthin, violaxanthin, zeaxanthin, antheraxanthin and cryptoxanthin) are more hydrophilic than carotenes. Plants such as various fruits and vegetables are the natural source of xanthophylls (Sommerburg et al., (1998) Br J Ophthalmol. 82: 907-910; Deli et al., (2001) J Agric Food Chem. 49:1517-23; Molnar et al., (2005) Phytother Res. 19:700-7) because xanthophylls play important roles in plant photoprotection by decreasing the possibility of cell damage caused by excess light (Demmig et al., (1987) Plant Physiol. 84:218-224; Niyogi et al., (1998) Plant Cell. 10:1121-34; Havaux and Niyogi, (1999) Proc Natl Acad Sci USA. 96:8762-7; Morosinotto et al., (2002) J Biol Chem. 277: 36913-20). It is interesting to note that xanthophylls play essential roles in visual health (Bone et al., (1988) Invest Ophthalmol Vis Sci. 29:843-849; Handelman et al., (1988) Invest Ophthalmol Vis Sci. 29:850-855; Yeum et al., (1995) Invest Ophthalmol Vis Sci. 36:2756-2761; Hammond et al., (1997) Invest Ophthalmol Vis Sci. 38:1795-1801; Johnson et al., (2000) Am J Clin Nutr. 71:1555-1562; Bone et al., (2003) J Nutr. 133: 992-998; Ribaya-Mercado and Blumberg, (2004) J Am Coll Nutr. 23 (6 Suppl):5675-5875). In addition, xanthophylls have been reported to be associated with lower risk of cancers (Orjuela et al., (2005) Cancer Epidemiol Biomarkers Prey. 14:1433-40; Kelemen et al., (2006) Am J Clin Nutr. 83: 1401-10; Zhang et al., (2007) Nutr Cancer. 59:46-53; Tamimi et al., (2009) Cancer Res. 69: 9323-9; Lee et al., (2009) Cancer Epidemiol Biomarkers Prey. 18:1730-9) and cardiovascular diseases (Street et al., (1994) Circulation 90:1154-1161; Dwyer et al., (2001) Circulation. 103:2922-7). The broad spectrum of biological activities described above suggests that xanthophylls are bioactive substances which are health benefits to the human beings.

Zeaxanthin, a member of the xanthophylls, is one of the essential components of the macular area. It should be noted that zeaxanthin can not be synthesized in animal bodies and therefore it must be obtained from the diet. For commercial purpose, zeaxanthin can be obtained via chemical synthesis (U.S. Pat. Nos. 4,952,716; 5,227,507) or via extraction from natural sources such as plants (U.S. Pat. Nos. 5,648,564; 6,784,351; 6,191,293; 7,150,890; 7,173,145) or microbes (U.S. Pat. Nos. 3,891,504; 3,951,742; 3,951,743; 5,308,759; 5427783). Due to the cost concern in the process of chemically synthesizing zeaxanthin and the health risk concern in the consumption of this artificial zeaxanthin, the natural zeaxanthin is preferential. Though it is reported that zeaxanthin can be extracted from natural sources shown as above prior art, the problems of low yield and low purity of zeaxanthin still exist and result in high cost of preparing zeaxanthin. Thus, a microorganism capable of producing high purity natural zeaxanthin is required.

### SUMMARY

The present invention provides a composition for prevention and/or treatment of human skin alteration or disease comprising an extract of marine bacteria selected from Flavobacteriaceae, wherein the extract contains an effective amount of zeaxanthin.

The present invention also provides a method for preparing the composition comprising an extract of marine bacteria selected from Flavobacteriaceae. wherein the extract contains an effective amount of zeaxanthin, which comprises: (a) culturing marine bacteria selected from Flavobacteriaceae, in a liquid culturing medium to form pigments containing zeaxanthin; (b) separating cell mass of the marine bacteria with the pigments and the liquid culturing medium and collecting the cell mass to obtain the composition, and (c) optionally mixing the cell mass and a carrier.

### BRIEF DESCRIPTION OF THE DRAWINGS

FIG. 1 shows the bacterial colonies (Olleya marilimosa VIG2317) on agar plate.

FIG. 2 shows the HPLC analysis of pure microbial zeaxanthin extracted from Olleya marilimosa VIG2317. The purity of the microbial zeaxanthin (F3; retention time:17.683 minutes) is greater than 90%.

FIG. 3 shows the photoprotection activity of the microbial zeaxanthin extracted from Olleya marilimosa VIG2317. After 15 minutes of UV-irradiation, the protective effect of the microbial zeaxanthin on the viability of human skin fibroblast cells (CCD-966SK cell line) was determined at 24 hours. The cell viability treated with the microbial zeaxanthin (20 μg/ml, 50 μg/ml) is increased 18.9% and 33.7%, respectively, as compared to that of no zeaxanthin treatment.

FIG. 4 shows the lipid peroxidation inhibitory activity of the microbial zeaxanthin, extracted from Olleya marilimosa VIG2317, determined using the ferric thiocyanate method at the concentration of 60 μg/ml. A comparison analysis demonstrates that the inhibitory activities (66.9%, 68.6% and 69.7%) are similar for zeaxanthin, lutein and β-carotene, respectively, at OD 500 nm.

FIG. 5 shows the lipid peroxidation inhibitory activity of the microbial zeaxanthin, extracted from Olleya marilimosa VIG2317, determined using the conjugated diene method at the concentration of 30 μg/ml. A comparison analysis demonstrates that the inhibitory activities (67.4%, 68.7% and 70.3%) are similar for zeaxanthin, lutein and β-carotene, respectively,at OD 234 nm.

FIG. 6 shows the lipid peroxidation inhibitory activity of the microbial zeaxanthin, extracted from Olleya marilimosa VIG2317, determined using the liposome-TBARS (Thiobarbituric acid-reactive substances) method. The percentages of inhibition on free MDA (malondialdehyde) formation determined at OD 532 nm are 55.23%, 61.05%, 71.16%, 78.45% and 80.75% for the microbial zeaxanthin at the concentrations of 125 μg/ml, 250 μg/ml, 500 μg/ml, 1000 μg/ml and 2000 μg/ml, respectively.

FIG. 7 shows the total antioxidant activity of the microbial zeaxanthin, extracted from Olleya marilimosa VIG2317, determined using the TEAC (trolox equivalent antioxidant capacity) method. The percentages of inhibition on absorbance determined at OD 734 nm are 15.68%, 28.13%, 53.35% and 91.22% for the microbial zeaxanthin at the concentrations of 62.5 μg/ml, 125 μg/ml, 250 μg/ml, and 500 μg/ml, respectively.

FIG. 8 shows the antioxidant activity of the microbial zeaxanthin, extracted from Olleya marilimosa VIG2317, determined using the DPPH (1,1-diphenyl-2-picrylhydrazyl) free radical scavenging method. The percentages of inhibition on absorbance determined at OD 517 nm are 10.02%, 13.04%, 30.18%, 48.20% and 96.69% for the microbial zeaxanthin at the concentrations of 62.5 μg/ml, 125 μg/ml, 250 μg/ml, 500 μg/ml and 1000 μg/ml, respectively.

FIG. 9 shows the effect of the microbial zeaxanthin, extracted from Olleya marilimosa VIG2317, on melanin content in B16/F10 melanoma cells. The percentages of inhibition on the melanin content determined at OD 400 nm are increased from 6.74% to 22.71% as the concentrations of the microbial zeaxanthin increased from 2 μg/ml to 20 μg/ml. When the incubation time was increased from 24 hrs to 48 hrs, percentages of inhibition on the melanin content are increased from 6.74% to 32.49% and from 22.71% to 36.74% for the concentrations of the microbial zeaxanthin at 2 μg/ml and 20 μg/ml, respectively.

FIG. 10 shows the anti-proliferation activity of the microbial zeaxanthin, extracted from Olleya marilimosa VIG2317, in human cancer cells using MTT assay. The cancer cell viabilities treated with 30 μg/ml and 60 μg/ml of natural zeaxanthin, respectively, are 74% and 67.7% for prostate carcinoma (PC-3); 72.3% and 75.3% for colorectal adenocarcinoma (LS123); 57.3% and 40.1% for gastric adenocarcinoma (AGS); 78.7% and 61.6% for breast adenocarcinoma (MCF7); 70.3% and 20.3% for ovarian cancer (TOV-112D); 72.6% and 28.5% for pharynx squamous cell carcinoma (FaDu); 75.6% and 33.4% for pancreatic adenocarcinoma (BxPC-3); 72.1% and 40.0% for choriocarcinoma (JAR); 45.9% and 16.4% for bladder primary carcinoma (5637) and 70.9% and 71.7% for thyroid squamous cell carcinoma (SW579). For comparison, the cell viabilities of non-cancer cells are 105.6% and 124.2% for retinal pigmented epithelium cell (ARPE-19); 102.3% and 101.2% for embryonic fibroblast (BCRC60118) and 95.8% and 99.8%f or human skin fibroblast (BCRC 60153).

### DETAILED DESCRIPTION OF THE EMBODIMENTS

According to the present invention, a composition comprising an extract of marine bacteria, containing zeaxanthin is provided. The invention further provides use of the same in the fields of nutraceutical, food additives, cosmetics/skin care and pharmaceuticals.

According to the present invention, the extract containing an effective amount of zeaxanthin is obtained from marine bacteria of the family Flavobacteriaceae. Therefore, the zeaxanthin extracted from marine bacteria of the family Flavobacteriaceae is the so-called “microbial zeaxanthin” in the present invention. The characteristics of marine bacteria of the family Flavobacteriaceae are gram-negative, yellow-pigmented, rod-shaped and aerobic. Many marine bacteria with the same characteristics are isolated from the family Flavobacteriaceae such as Zeaxanthinibacter enoshimensis (Asker et al., (2007) Int J Syst Evol Microbiol. 57(Pt 4):837-43), Olleya marilimosa (Nichols et al., (2005) Int J Syst Evol Microbiol. 55(Pt 4):1557-61), Paracoccus zeaxanthinifaciens (Berry et al., (2003) Int J Syst Evol Microbiol. 53(Pt 1):231-8), and Hyunsoonleella jejuensis (Yoon et al., (2010) Int J Syst Evol Microbiol 60(Pt 2):382-6).

In one embodiment of the present invention, the marine bacteria is selected from a group consisting of Zeaxanthinibacter, Olleya, Paracoccus, Hyunsoonleella, and mutants thereof.

In another embodiment of the present invention, the marine bacteria are selected from a group consisting of Zeaxanthinibacter enoshimensis, Olleya marilimosa, Paracoccus zeaxanthinifaciens, Hyunsoonleella jejuensis and mutants thereof.

According to the present invention, the marine bacterium is Olleya marilimosa. More specifically, the marine bacterium is a novel strain, Olleya marilimosa VIG2317, having Accession Deposit Number CCTCC M2010201, which was deposited at the China Center for Type Culture Collection.

According to the present invention, the composition comprising marine bacterial extract obtained from Olleya marilimosa VIG2317 contains from about 0.1 mg to about 10 mg as effective amount of microbial zeaxanthin. In one preferred embodiment, the effective amount is about 0.5 mg to about 10 mg.

According to the present invention, and the purity of the microbial zeaxanthin obtained from Olleya marilimosa VIG2317 can be at least 90% (as determined by HPLC area %).

The composition of the present invention can be used to treat or prevent human skin alteration, such as skin aging resulted from exposure to UV radiation and/or skin pigmentation resulted from increased melanin content.

The composition of the present invention can be used to treat or prevent diseases such as eye diseases, cardiovascular diseases, or cancers. In one embodiment, the eye diseases are caused by insufficient levels of macular zeaxanthin and/or serum zeaxanthin. In one embodiment, the cardiovascular diseases are associated with increased lipid peroxidation and/or malondialdehyde. In one embodiment, the cancers are prostate carcinoma, colorectal adenocarcinoma, gastric adenocarcinoma, breast adenocarcinoma, ovarian cancer, pharynx squamous cell carcinoma, pancreatic adenocarcinoma, choriocarcinoma, bladder primary carcinoma, and/or thyroid squamous cell carcinoma.

The composition of the present invention comprises the marine bacterial extract containing microbial zeaxanthin, and a suitable carrier or excipient, wherein the carrier is, for example but not limitation, water, oil, organic solvent and the like. The composition of the present invention can be administered topically or orally.

The present invention also provides a method for preparing the above composition containing microbial zeaxanthin, comprising the steps of: (a) culturing marine bacteria selected from Flavobacteriaceae in a liquid culturing medium to form pigments containing zeaxanthin; (b) separating cell mass of the marine bacteria with the pigments and the liquid culturing medium and collect the cell mass to obtain the composition containing microbial zeaxanthin, and (c) optionally mixing the cell mass and a carrier. The composition can be directly used as food additive or nutrient without further purification.

In one embodiment, the present invention further comprises the following steps: (d) lysing the marine bacteria by pulverizing or otherwise, and optionally further digesting the cell debris if needed, and (e) dissolving the pigments by using a solvent; and (f) removing the solvent to obtain zeaxanthin. Of course other purification steps can be used or added thereto, including various recrystallization, precipitation, supercritical extraction, or chromatography steps, as desired. From above steps, the microbial zeaxanthin can be further isolated and purified, but zeaxanthin obtained from step (f) has at least 90% (HPLC area %) purity. In another embodiment, the purity is at least 95%, or at least 97%.

By “lysing” what is meant herein is any method of breaking open the bacteria. Thus, the cells can be freeze dried, ground or pulverized, opened with heat or chemicals, or biologically lysed with enzymes or changes in osmolality, or combinations and variations thereof.

Solvents that can be used in the invention include any solvent or combination thereof, that will preferentially dissolve the yellow zeaxanthin pigment. Such solvents include, but are not limited to acetone, methanol, ethanol, ethyl acetate, isopropylalcohol , cyclohexane, and mixtures and combinations therof. Many solvent systems have been used to separate various planty poigments, and these systems are adaptable to use with the cell extracts produced herein.

According to the method of the present invention, the marine bacteria is selected from a group consisting of Zeaxanthinibacter, Olleya, Paracoccus,. Hyunsoonleella, and mutants thereof More particulary, the marine bacteria is selected from a group consisting of Zeaxanthinibacter enoshimensis, Olleya marilimosa, Paracoccus zeaxanthinifaciens,. Hyunsoonleella jejuensis, and mutants thereof.

In one embodiment of the present invention, the marine bacterium is selected from a group consisting of Olleya marilimosa and mutants thereof More particularly, the marine bacterium is Olleya marilimosa VIG2317 having Accession Deposit Number CCTCC M2010201.

### EXAMPLES

The following steps are used for extracting zeaxanthin from Olleya marilimosa VIG2317.

1. Bacterial Characterization and Culture.

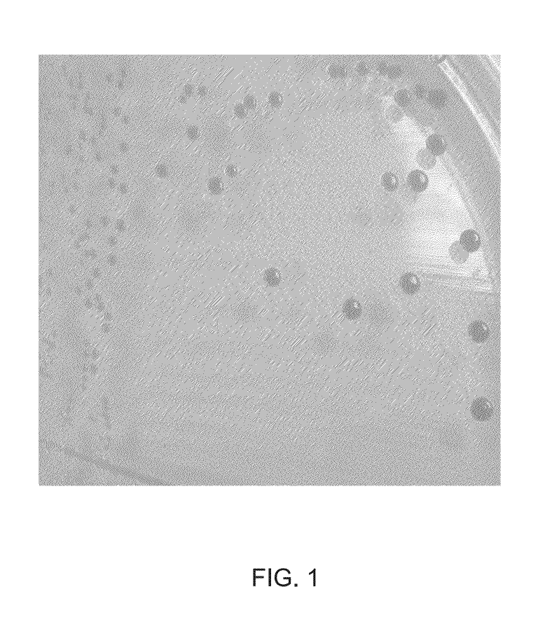

A bacterial strain (Olleya marilimosa VIG2317 having Accession Deposit Number CCTCC M2010201), isolated from a sea water sample collected from the Pacific Ocean on the east coast of Taiwan, formed yellow colonies on marine agar (FIG. 1) that had been incubated at 25° C. for 2 days. The characterization of the strain VIG2317 was carried out by the Food Industry Research and Development Institute (FIRDI), Hsinchu, Taiwan. The cells are gram-negative, yellow-pigmented, rod-shaped, motile, aerobic and non-endospore-forming According to 16S rRNA gene sequence analysis, strain VIG2317 was closely related (99% sequence similarity) to Olleya marilimosa. Biochemical and morphological characteristics of VIG2317 and Olley marilimosa CIP 108537 is shown in TABLE 1.

Individual colonies of VIG2317 on the plates were picked and cultured in rich media such as marine broth in the flask, which used as seed culture for the fermentation. A volume of seed culture was transferred to fermenter.

TABLE 1

| Characteristic | VIG2317 | Olley marilimosa CIP 108537^{T} |
|---|---|---|
| Growth at 25° C. | + | + |
| Growth at 30° C. | + | + |
| Motility | + | + |
| Production of: | | |
| Pigment | + | + |
| H₂S | − | − |
| Indole | − | − |
| β-galactosidase | − | − |
| Urease | − | − |
| Oxidase | + | + |
| Catalase | + | + |
| Acetoin | + | − |
| Arginine dihydrolase | − | − |
| β-glucosidase | + | − |
| Degraded of gelatin | + | + |
| Nitrate reduction | − | − |
| Assimilated of: | | |
| Glucose | + | + |
| Maltose | + | + |
| Mannose | + | + |
| Arabinose | − | − |
| Mannitol | − | − |
| D-gluconate | − | − |
| Capric acid | − | − |
| Adipic acid | − | − |
| Trisodium citrate | − | − |

2. Extraction, Purification and HPLC Analysis of Microbial Zeaxanthin

Sample culture harvested from the fermenter was extracted with solvent such as acetone. The acetone extract was applied onto a silica gel column and eluted with a mixture of ethyl-acetate to hexane to 3:7(v/v). Using freeze dryer, the collected fraction was powdered for high-performance liquid chromatography (HPLC) analysis.

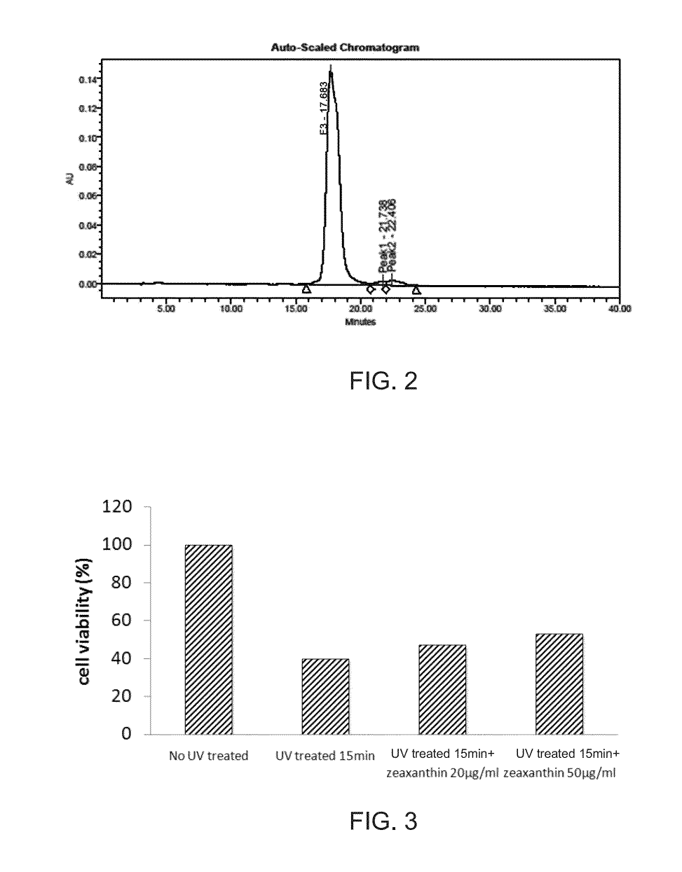

The dried extract was dissolved in methanol and filtered for HPLC analysis. The HPLC system was programmed to inject 10 μl samples into the 4.6×250 mm ODS C-18 column. The mobile phase contained 20% methanol, 73% acetonitrile, 7% Tris-HCl buffer. The column was operated at room temperature. Separation was carried out at a flow rate of 1.0 ml/min. The detection wavelength was 450 nm. The purity of microbial zeaxanthin was greater than 90% analyzed using HPLC (FIG. 2).

3. Determination of Photoprotection Activity of Microbial Zeaxanthin

Human skin fibroblast cells (CCD-966SK cell line) were cultured on the dish. The cells were treated with microbial zeaxanthin dissolved in dimethyl sulfoxide. After treated for 24 hours the dishes were irradiated UV for 15 min and the cells were incubated for 24 hours. The cells were detached by trypsin and counted by hemocytometer. The protection effect of microbial zeaxanthin on the viability of fibroblast cells is provided (FIG. 3).

Skin fibroblast cells, the essential cells of skin, are responsible for the production of collagen, a structural component of the skin (Stanley et al., (1985) J Invest Dermatol. 85:542-545; Olsen et al., (1989) J. Clin. Invest. 83: 791-795; Akagi et al., (1999) J. Invest. Dermatol. 113: 246-250). Collagen reduction is a biological phenomenon responsible for the wrinkled appearance found in the aged skin caused either intrinsic (chronologic aging) or extrinsic (e.g., photoaging: exposure to UV irradiation) (Burke et al., (1994) Exp Gerontol 29:37-53; Fisher et al., (1997) New Eng J Med 337:1419-1428; Fligiel et al., (2003) J Invest Dermatol. 120:842-848; Varani et al., (2006) Am J Pathol. 168:1861-8; Chauhan and Shakya, (2009) Indian J Dermatol Venereol Leprol. 75:463-8). In addition, UV irradiation has been reported to cause loss of cell viability (Kulms and Schwarz (2000) Photodermatol Photoimmunol Photomed.16:195-201; Murphy et al., (2001) Exp. Dermatol. 10:155-160; Ichihashi et al., (2003) Toxicology. 189:21-39; Philips et al., (2007) Arch Dermatol Res. 299:373-9). After UV irradiation, the viability of human skin fibroblast cells treated with microbial zeaxanthin (20 μg/ml, 50 μg/ml) is increased 18.9% and 33.7%, respectively, as compared to that of no microbial zeaxanthin treatment. It should be noted that this increased cell viability is associated with the increased concentration of microbial zeaxanthin suggesting that microbial zeaxanthin is an important photoprotective agent which can be used in the fields of nutraceutical, food additives, cosmetics/skin care and pharmaceuticals.

4. Determination of Antioxidant Activities of Microbial Zeaxanthin

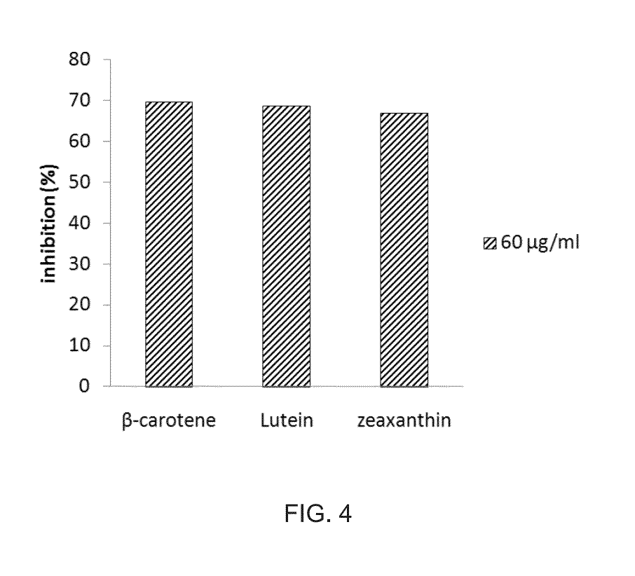

The antioxidant activities of microbial zeaxanthin are provided (FIGS. 4-7). Since carotenoids are a group of organic pigments known to reduce the health risks of the biological systems via their antioxidant activities (Burton, (1989) J Nutr. 119: 109-11), it is thus essential to demonstrate if the microbial zeaxanthin is a bioactive ingredient in preventing cell damage caused by oxidation chain reactions.

(1) Lipid peroxidation inhibitory activity of the microbial zeaxanthin determined using the ferric thiocyanate method (FIG. 4):

Microbial zeaxanthin was dissolved in 0.2 M potassium phosphate buffer and mixed with linoleic acid emulsion mixture prepared by potassium phosphate buffer. After 24 hours incubation in 37′C, the mixture was added with 75% ethanol, ammonium thiocyanate, and iron(II) chloroide tetrahydrate. The absorbance at 500 nm was determined after 1 min incubation. Inhibtion activity=[1−(Abs of sample/Abs of blank)]*100%. A comparison analysis demonstrates that the inhibitory activities (66.9%, 68.6% and 69.7%) are similar for zeaxanthin, lutein and β-carotene, respectively.

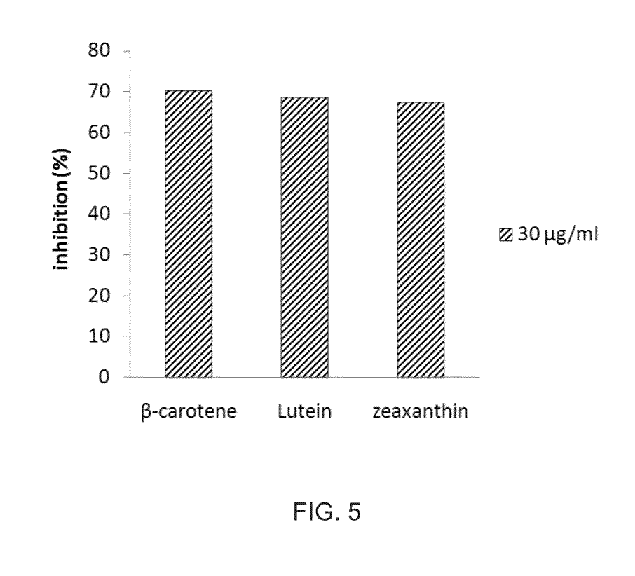

(2) Lipid peroxidation inhibitory activity of the microbial zeaxanthin determined using the conjugated diene method (FIG. 5):

Microbial zeaxanthin was dissolved in 0.2 M potassium phosphate buffer and mixed with linoleic acid emulsion mixture prepared by potassium phosphate buffer. The absorbance of 234 nm was determined after 15 hour incubation in 37° C. Inhibition activity=[1−(Abs of sample/Abs of blank)]*100%. A comparison analysis demonstrates that the inhibitory activities (67.4%, 68.7% and 70.3%) are similar for zeaxanthin, lutein and β-carotene, respectively.

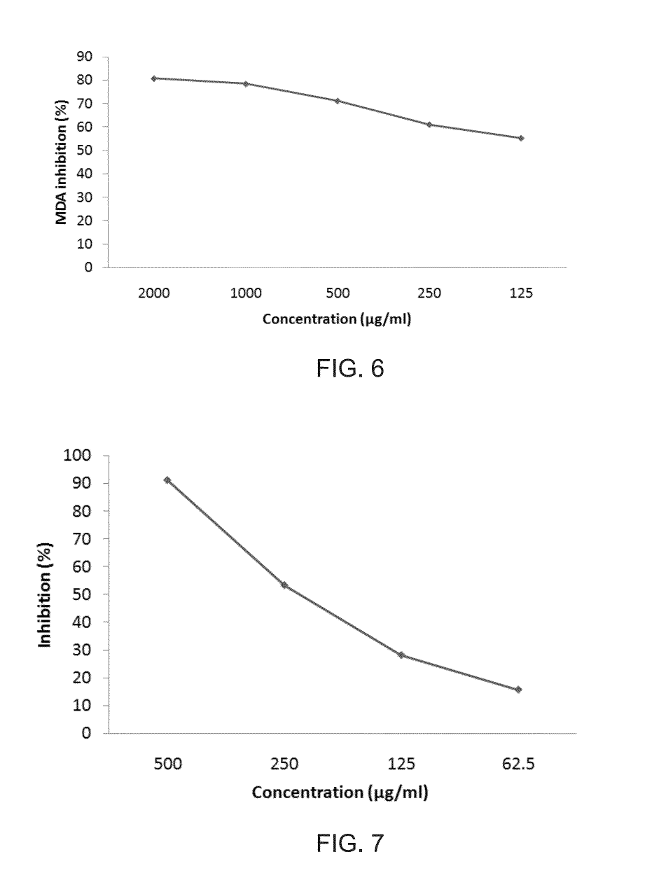

(3) Lipid peroxidation inhibitory activity of the microbial zeaxanthin determined using the liposome-TBARS (Thiobarbituric acid-reactive substances) method (FIG. 6).

Microbial zeaxanthin was dissolved in methanol and mixed with liposome emulsion, iron(III) chloride, and ascorbic acid. After 2 hours water bath in 37° C. the mixture was added with butylated hydorxytoluene, thibarbituric acid, and trichloroacetic acid. After 20 min of water bath (100° C.), the mixture was chilled with ice. The absorbance at 532 nm was determined MDA (malondialdehyde) inhibiton activity=[1−(Abs of sample/Abs of blank)]*100%. The percentages of inhibition on free MDA are increased from 55.23% to 80.75% as the concentrations of the microbial zeaxanthin increased from 125 μg/ml to 2000 μg/ml.

(4) The antioxidant activity of the microbial zeaxanthin determined using the TEAC (trolox equivalent antioxidant capacity) method (FIG. 7).

Microbial zeaxanthin was dissolved in 0.01M sodium phosphate buffer and mixed with 2,2′-azino-bis(3-ethylbenzthiazoline-6-sulphonic acid) containing potassium persulfate. The absorbance at 734 nm was determined after 30 min incubation. Inhibition activity=[1−(Abs of sample/Abs of blank)]*100%. The percentages of inhibition are increased from 15.68% to 91.22% as the concentrations of the microbial zeaxanthin increased from 62.5 μg/ml to 500 μg/ml.

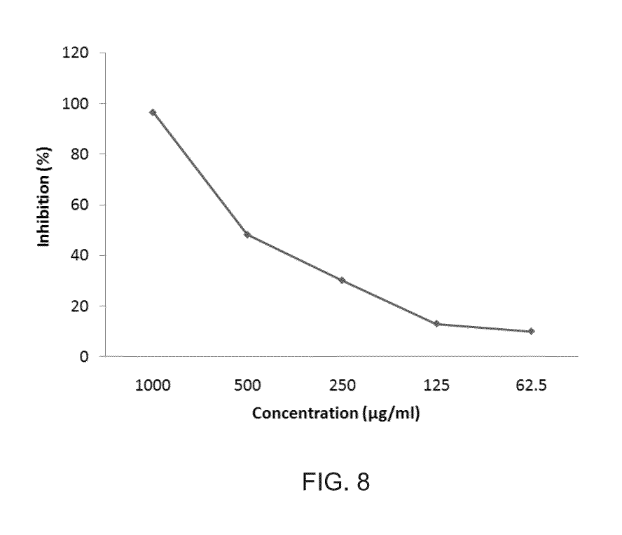

(5) The antioxidant activity of the microbial zeaxanthin determined using the DPPH (1,1-diphenyl-2-picrylhydrazyl) free radical scavenging method (FIG. 8).

Microbial zeaxanthin was dissolved in methanol and mixed with 2,2-diphenyl-1-picrylhydrazyl in methanol. The absorbance at 517 nm was determined after 30 min incubation. Inhibition activity=[1−(Abs of sample/Abs of blank)]*100%. The percentages of inhibition are increased from 10.02% to 96.69% as the concentrations of the microbial zeaxanthin increased from 62.5 μg/ml to 1000 μg/ml. These results suggest that the microbial zeaxanthin is a potent antioxidant, which can be used in the fields of nutraceutical, food additives, cosmetics/skin care and pharmaceuticals.

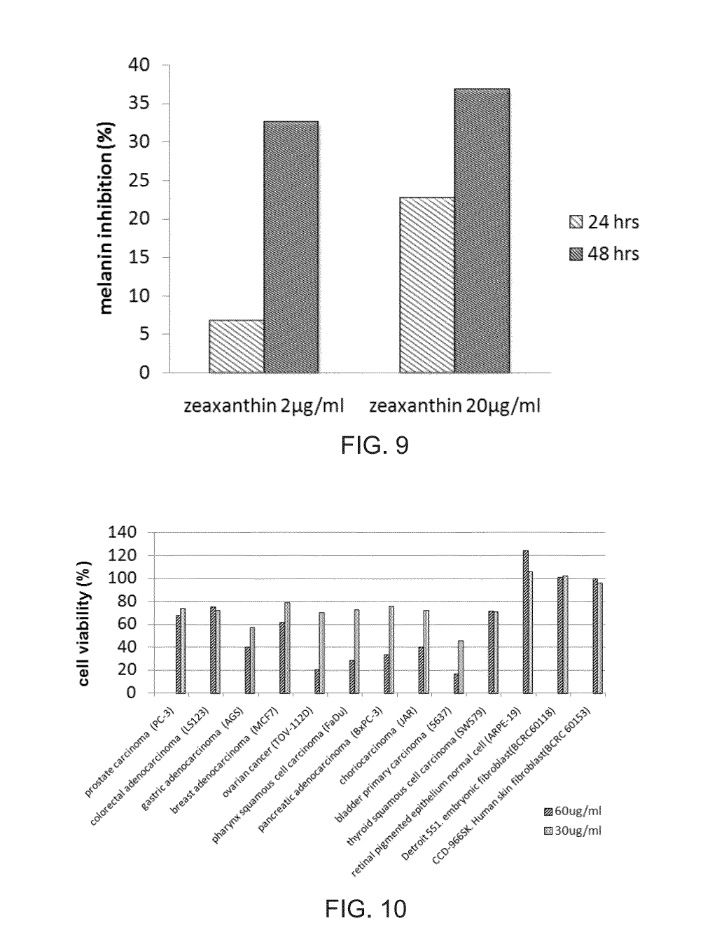

5. The Effect of the Microbial Zeaxanthin on Melanin Content in B16/F10 Melanoma Cells (FIG. 9).

B16 F0 mouse melanoma cells were cultured on the dish. The cells were treated with microbial zeaxanthin dissolved in dimethyl sulfoxide. After treated for 24 hrs and 48 hrs, respectively, the medium was removed and the cells were washed with PBS and then dissolved in 1 N NaOH containing 10% DMSO. The relative melanin content was determined at OD400 nm. The percentages of inhibition on melanin content are increased from 6.74% to 22.71% as the concentrations of the microbial zeaxanthin increased from 2 μg/ml to 20 μg/ml. When the incubation time is increased from 24 hrs to 48 hrs, the inhibition are increased from 6.74% to 32.49% and from 22.71% to 36.74% for the concentrations of the microbial zeaxanthin at 2 μg/ml and 20 μg/ml, respectively. The results indicate that the microbial zeaxanthin causes dose- and time-dependent inhibiton on melanin content. This finding suggests that the microbial zeaxanthin can be used as whitening agent for cosmetics use.

6. The Anti-Proliferation Activity of the Microbial Zeaxanthin on Cancer Cells (FIG. 10).

Cancer cells were cultured on a dish, and the cells were treated with microbial zeaxanthin dissolved in dimethyl sulfoxide. After 24 hrs incubation, thiazolyl blue tetrazolium bromide was added to the cells and incubated for 4 hours. The medium was removed and the crystals were dissolved in dimethyl sulfoxide followed by measuring the absorbance at 590 nm. Cell viability=(Abs of cell treated zeaxanthin/Abs of cell with no treatment)*100%. Since xanthophylls have been reported to be associated with lower risk of cancers (Orjuela et al., (2005) Cancer Epidemiol Biomarkers Prey. 14:1433-40; Kelemen et al., (2006)_Am J Clin Nutr. 83: 1401-10; Zhang et al., (2007) Nutr Cancer. 59:46-53; Tamimi et al., (2009)_Cancer Res. 69: 9323-9; Lee et al., (2009) Cancer Epidemiol Biomarkers Prey. 18:1730-9), it is necessary to determine if the microbial zeaxanthin contains anti-cancer activity. Using 30 μg/ml of the microbial zeaxanthin, the cell viabilities are 74% for prostate carcinoma (PC-3); 72.3% for colorectal adenocarcinoma (LS123); 57.3% for gastric adenocarcinoma (AGS); 78.7% for breast adenocarcinoma (MCF7); 70.3% for ovarian cancer (TOV-112D); 72.6% for pharynx squamous cell carcinoma (FaDu); 75.6% for pancreatic adenocarcinoma (BxPC-3); 72.1% for choriocarcinoma (JAR); 45.9% for bladder primary carcinoma (5637) and 70.9% for thyroid squamous cell carcinoma (SW579). For comparison, the cell viabilities of non-cancer cells are 105.6% for retinal pigmented epithelium cell (ARPE-19); 102.3% for embryonic fibroblast (BCRC60118) and 95.8% for human skin fibroblast (BCRC 60153). Under the condition of increasing the microbial zeaxanthin concentration to 60 μg/ml, the cell viabilities are 67.7% for prostate carcinoma (PC-3); 75.3% for colorectal adenocarcinoma (LS123); 40.1% for gastric adenocarcinoma (AGS); 61.6% for breast adenocarcinoma (MCF7); 20.3% for ovarian cancer (TOV-112D); 28.5% for pharynx squamous cell carcinoma (FaDu); 33.4% for pancreatic adenocarcinoma (BxPC-3); 40.0% for choriocarcinoma (JAR); 16.4% for bladder primary carcinoma (5637) and 71.7% for thyroid squamous cell carcinoma (SW579). For comparison, the cell viabilities of non-cancer cells are 124.2% for retinal pigmented epithelium cell (ARPE-19); 101.2% for embryonic fibroblast (BCRC60118) and 99.8% for human skin fibroblast (BCRC 60153). The results indicate that all cancer cells tested in the present invention are sensitive to the microbial zeaxanthin at the concentration of 30 μg/ml. However, it should be noted that dose-dependent suppression of cell proliferation is found in some cancer cells such as ovarian cancer, pharynx squamous cell carcinoma, pancreatic adenocarcinoma, choriocarcinoma, and bladder primary carcinoma when the concentration of the microbial zeaxanthin is increased to 60 μg/ml. These results suggest that the microbial zeaxanthin is an anti-cancer agent which can be used in the fields of nutraceutical, food additives, cosmetics/skin care and pharmaceuticals.

The following formulations are provided for illustration, but not for limiting the invention.

Formulation 1: Essence

Microbial zeaxanthin (400 ppm in butylene glycol) is mixed with sodium metabisulfite, ethylhexylglycerin, phenoxyethanol, arginine, dipotassium glycyrrhizate, sodium hyaluronate, PEG-16 macadamia glycerides, acrylates/C10-30 alkyl acrylate crosspolymer, disodium EDTA and water.

Formulation 2: Milk Lotion

Microbial zeaxanthin (400 ppm in Butylene Glycol) is mixed with sodium metabisulfite, citric acid, sodium citrate, tocopheryl acetate, cellulose gum, potassium sorbare, ethylhexylglycerin, bisabolol, ammonium acryloyldimethyltaurate/vp copolymer, butyrospermum parkii (shea butter), beheneth-25, cetyl alcohol, glycerin, cetearyl olivate, isononyl isononanoate, helianthus annuus (sunflower) seed oil, phenoxyethanol, sodium hyaluronate, disodium EDTA and water.

Formulation 3: Dietary Supplements/Pharmaceutical Dosage

Microbial zeaxanthin 10 mg is dissolved in corn oil and packaged in the form of soft gelatin capsule.

Formulation 4: Dietary Supplements/Pharmaceutical Dosage

Microbial zeaxanthin 10 mg is mixed with granulating agents and packaged in the form of tablet.

Formulation 5: Dietary Supplements

Microbial zeaxanthin 10 mg is mixed with lutein and/or other nutrients and/or antioxidants dissolved in corn oil/peanut oil and packaged in the form of soft gelatin capsule.

Formulation 6: Dietary Supplements

Microbial zeaxanthin 10 mg is mixed with lutein and/or other nutrients and/or minerals and packaged in the form of tablet.

What is claimed is:

1. An isolated bacteria strain capable of producing zeaxanthin identified as Olleya marilimosa having Accession Deposit Number CCTCC M2010201.

2. The bacteria strain according to claim 1, wherein a separated cell mass of the bacteria strain containing the pigments has a purity of zeaxanthin that is at least 90% (high performance liquid chromatography area %).
